(19) United States
(12) Patent Application Publication
(10) Pub. No.: US 2008/0071224 A1
(43) Pub. Date: Mar. 20, 2008

(54) Catheter/IV cover

(76) Inventor: Barbara B. Forsyth, St. Louis, MO (US)

Correspondence Address: Paul M. Denk, Suite 170, 763 S. New Ballas Rd., St Louis, MO, 63141, US

(21) Appl. No.: 11/900,095

(22) Filed: Sep. 9, 2007

Related U.S. Application Data
(60) Provisional application No. 60/844,436, filed on Sep. 14, 2006.

(51) Int. Cl. A61M 5/32

(52) U.S. Cl. 604/179; 604/174

(57) ABSTRACT

A catheter/IV cover including a covering portion, having at least one loop extending from one side of the cover, a strap of flexible material extending from the opposite side of the covering material, with the strap and loops capable of engagement, through any type of fastener, Velcro®, or buckle, when securing the catheter/IV cover in place about the arm, the wrist, or the hand. There may be provided an aperture through the covering portion, which allows for viewing of the underlying and emplaced catheter, and its tubing, or there may be a mesh material, or other material of softness and transparency, to allow for viewing of the catheter when in place, and which allows flexibility when covered by this cover, which also allows for freedom of bending of the hand, or the arm at the elbow, while the cover is affixed.

### CROSS REFERENCE TO RELATED APPLICATION

This non-provisional application claims priority to provisional application Ser. No. 60/844,436 filed on Sep. 14, 2006.

### FIELD OF INVENTION

This particular invention relates to a catheter/IV cover that has special benefits for the ambulatory patient receiving intravenous therapy, and which cover is intended to protect the catheter and the loose and exposed tubing leading up to the catheter, with the cover having capability of being applied to the region of the arm, in the vicinity of the elbow, or above and below thereof, and the wrist area of the arm, and even for covering a catheter upon the hand.

### BACKGROUND OF THE INVENTION

This invention does relate to a catheter/IV cover. Numerous types of straps or other devices have been devised for facilitating the application of a catheter cover, usually in the region of the arm, but such covers normally simply provide some form of coverage, but do not facilitate the retention of the catheter in place, particularly for an active patient, or one which may be moved to different regions of the hospital, or the rest home, or returning home, during treatment and convalescence.

For example, the patent to Rayhart, U.S. Pat. No. 3,160,158, shows a Support for Catheter and the Like. This device includes a snap pin that applies to the arm, and incorporates a strip that can be clasped on top of the closed strap, apparently for holding a catheter or other medical instrument in location.

The patent to Duncan, U.S. Pat. No. 4,481,942, shows an Infant Arm Restraint. This arm restraint is for application around the elbow of an infant to restrain movement of the infant's hands, to certain areas.

The patent to Turner, U.S. Pat. No. 4,531,942, shows an IV Securing Means. This device apparently is for securement to the hand, and includes a thumb opening, and incorporates a series of fibrous loop materials, when it is wrapped around the hand during usage. This is apparently for holding a needle in place.

The patent to Christie, U.S. Pat. No. 4,91,356, shows another Intravenous Needle Stabilizing Band. This device includes a band incorporating a strip, that applies to the arm, through the use of Velcro components, and includes a separate strip for securement of the tube part of the catheter to the band. This holds the tube part in position during usage.

The patent to Widman, U.S. Pat. No. 4,671,787, shows another Support Wrap System for Intravenous Tubing. This tubing is also applied to, in this instance, apparently near the elbow, or within its region, and wraps entirely around the arm, for locating of the IV tube, and its needle, in place.

The patent to Fritts, U.S. Pat. No. 5,188,608, shows another Protective Stabilizing Sleeve for IV Needle. The sleeve can be applied directly to the arm, in the location where the needle and its tube locate. As can be seen, the layered sleeve, which may be formed of cloth, polyester, or an elastic blend, are designed for overlying the IV device, to keep it from unauthorized removal or pulling free from the patient's arm.

The patent to Bird, et al., U.S. Pat. No. 5,352,209, shows another Band for Anchoring a Tubular Device to the Body. This is another type of a primary strap for securement of about a portion of the body, such as the leg, and has a supplemental strap portion for holding the catheter in place.

The patent to Shesol, et al., U.S. Pat. No. 5,897,519, shows another Intravenous Securing Device and Secondary Wound Dressing. This device apparently functions as a means for holding a catheter in place, or at least its tubing, but it also incorporates what appears to be a pad or gauze for use in treatment of the wound area.

The patent to Villapiano, U.S. Pat. No. 6,032,289, shows a Security Garment, incorporating fastening straps.

The design patent to Inglish, U.S. Pat. No. D453,831, shows an IV Cover. It is a form of cover that has loops at one end, and buttons at the other, for securement of the cover about the arm.

The patent to Wilke, U.S. Pat. No. 6,464,669, shows another Catheter Protector. This device includes a method of protecting a catheter that includes a step of inserting the catheter into the patient, and then covering the inserted site with this protector.

The patent to Rozier, et al., U.S. Pat. No. 6,526,981, is upon a Site Guard for Intravenous Sites and Other Sensitive Areas. It defines a site guard that incorporates a hollow member having a base, and a fabric connector that affixes to the edge of the hollow member.

The patent to Bird, et al., U.S. Pat. No. 6,645,185, shows another Band for Anchoring Tubular Device to the Body. This is another band, that apparently has elasticity applied to it, and which incorporates a slip resistant material, as it is applied about the leg.

There are also a number of published applications that have been issued by the Patent Office. For example, the published application US 2003/0055382, to Schaeffer, is upon an Intravenous Catheter Support. The essence of this invention sets forth a support incorporating a base member, with its tube holding loops or hooks and through which the various catheter tubing can be applied.

The publication to Rose, US 2004/0138623, shows another Device for Securing Intravenous Needles to Treatment Sites. This device is primarily used for holding pads in place, through the use of straps, that are identified and shown as crisscrossing over each other to hold the catheters in place.

The published application to Jenkins, US 2005/0133043, shows an Arm Immobilizer. This is a closeable sleeve of compressible material placed around the patient's arm above or below the elbow joint, and designed for holding various catheters in place at an intravenous site.

The published application to Walsh, et al., US 2005/0137496, shows a pad or base with its strap held by Velcro, though the use of a series of secondary straps, for holding the transducer, IV, or other assemblies in place.

There are a variety of other types of devices that are used for holding drug infusion and delivery systems in place, many of them have been reviewed in the foregoing patents and publications, and there is also a U.S. Pat. No. 6,257,240, upon a Combination Protective Medical Guard with Self-Contained Supports. As shown in this patent, it is for use for holding medical devices or for protecting a surgical site, and incorporates a top port for use for viewing the site through the top of the protective guard.

U.S. Pat. No. 5,167,240, shows another Infusion Site Guard. These are protective devices.

There are also certain publications that show various types of mid-arm protectors, such as the product from Brown Medical, and which provides a type of thermoplastic sleeve that acts as a covering for the dressing area. It provides a water-proof seal to the area.

There are also various types of gauze coverings, that are applied as wraps to the surgery area, to act as an IV cover. This can be seen in the Cystic-L Device.

### SUMMARY OF THE INVENTION

The current invention provides what are believed to be enhancements and improvements over the types of covers as previously reviewed.

The current invention provides a patch like cover that is applied to ambulatory patients, which require the infusion of intravenous therapy, while convalescing, and which incorporates a resilient material, held by straps about the patient's arm, wrist, or hand, and which includes a window proximate which the various catheter(s) may locate, held in position during usage, and which also facilitates the bending of the arm during application, when the cover is applied in the region of the elbow.

In one embodiment of the invention, the cover is formed of a resilient material, preferably from a material identified as the Ultrasuede type, is of a rectangular configuration to provide for coverage at the vicinity where the catheter is applied, and to furnish means for holding of the catheter and its tubing in place, during usage. Ultrasuede is available from Toray UltraSuede, Inc., located in the State of New York. In this manner, it adds more permanence to the location of the catheter, and does not allow for its easy inadvertent disengagement during application.

In order to hold the cover in place, a series of lateral straps append to the cover, along one side, while the opposite side includes a series of loops, slits within tabs, or the like, and through which the straps may locate, and to be fastened either by Velcro, a buckle, or other means for securement. Velcro is available from Velcro Industries B.V., located in Amsterdam, Netherlands. The open portion of the cover may be to any dimensions, that allows for disclosure of the catheter therethrough, and also is provided for making the cover more susceptible to bending, at that location, in the region of the elbow, when the patient moves his/her arm in place. The cut out segment may be an oval cut, or to other configuration, and may be covered with a soft material, such as polyester tricot, which helps to retain the catheter in place, but at the same time, such material may be moderately transparent, so that the instrument can be also viewed, in place, to assure that it is maintaining its proper installation. In addition, there may be shaped guides affixed to the ends of the straps in order to facilitate their insertion through the various rings or loops, when the cover is being installed.

The best mode for connection of the straps to the Ultrasuede cover is to provide an extending tab from the Ultrasuede cover at the location where each strap is to be affixed, provide a slit through that tab, and then extend the end of the strap through the slit, fold back the tab under the cover overlaying the end of the strap, and then stitch the entire combination together to furnish a fully reinforced connection of a strap to the cover during its assembly.

The invention also contemplates the construction of similar types of covers, formed of the same type of resilient material, such as the Ultrasuede, and in the instance of the usage of the cover for application to the lower arm, or wrist, may have a cover segment that is of lesser square or rectangular dimensions than the cover as previously reviewed. Nevertheless, the cover will be fabricated of resilient material, so that it can adequately cover the instruments used in intravenous therapy, and assure that the catheter remains in place, when applied about the wrist area, during application and is comfortable to wear. Because of the fabrication of this wrist cover of the Ultrasuede material, the softness of such material is far more comfortable to the wearer, than any type of catheter/IV cover currently available. In this instance, since the cover is fabricated of a soft and resilient material that is of smaller dimensions, fewer straps may be required to secure the cover in place. Hence, in this embodiment, there may be a pair of straps that laterally extend from the one side of the cover, and can be applied through loops or slots at the opposite side of the cover, when the patch cover is applied in place. The straps and loops may be fabricated of similar materials to those as previously summarized, in order to provide for their effective usage in holding the various components in place.

A third embodiment for the cover, for use for overlying a catheter, is when it is applied into the region of the hand. In this instance, the cover will resemble the shape of the back of the hand, and have holes in its upper corners for application to the thumb and the little finger, with a strap provided at the bottom edge, for looping about the back of the hand, and securement, to provide a resilient protective cover over the catheter when it is applied in the region of the hand. Such a cover could be applied to the back of the hand, or also to the palm of the hand, during its usage, if this becomes necessary. It also is fabricated from a resilient soft material, but yet function as a structural cover for retention of the catheter in place, once installed, and applied, to assure that unauthorized removal or inadvertent disengagement does not take place.

It is, therefore, the principal object of this invention to provide a resilient patch cover for ambulatory patients receiving intravenous therapy to assure that the catheter and delivery tubing remains in place, upon the arm, wrist, or hand, and is not inadvertently disengaged.

Another object of this invention is to provide a catheter cover which is formed of a very soft and resilient material which is sufficiently sturdy to assure that the catheter and its operative components are held in place, but at the same time does not add to the discomfort of the patient.

Still another object of this invention is to provide a catheter/IV cover that has a cut out segment provided therein, and through which the catheter and tube may be observed, but also furnishes a location where the arm can be conveniently bent, as at the elbow, without any obstruction.

Still another object of this invention is to provide a catheter/IV cover that allows and maintains freedom of movement and comfort for the patient, during its usage and application.

Still another object of this invention is to provide a catheter/IV cover which has sufficient resiliency, and permeability, so as to allow air to circulate and to attain movement to lessen any generation of perspiration during usage.

Yet another object of this invention is to provide a catheter cover that allows the usage of normal clothing, to be worn without having to compensate for the presence of the application of the device.

Still another object of this invention is to provide a catheter/IV cover that can be worn while the patient is awake and active, or while he/she sleeps.

Still another object of this invention is to provide a catheter/IV cover that can be fabricated of various colorations, so as to add to the enjoyment and comfort of their usage and application.

It is still another object of this invention to provide a catheter/IV cover that is easy to maintain, and has enhanced washability attributes.

An important object of this invention is to provide a catheter/IV cover fabricated of select materials, and depending upon their assembly, can be used in the region of the elbow, the arm, the wrist, and upon the hand.

These and other objects may become more apparent to those skilled in the art upon review of the summary of the invention as provided herein, and upon undertaking a study of the description of its preferred embodiment, in view of the drawings.

### BRIEF DESCRIPTION OF THE DRAWINGS

In referring to the drawings,

FIG. 1 discloses a catheter/IV cover of this invention in place approximate the elbow region of the arm of the patient;

FIG. 2 shows a plan view of a catheter/IV cover for the upper arm;

FIG. 3 a-d discloses the catheter/IV cover in its various perspective views and in application on the upper arm;

FIG. 4 shows the catheter/IV cover, as modified, with a cut out segment to add flexibility to the cover during application, and to perhaps provide a viewing area to the region of the emplaced catheter on the upper arm;

FIG. 5 is an exploded view of the various components that make up the upper arm catheter/IV cover of FIG. 4;

FIG. 6 shows the catheter/IV cover, as modified, and applied to the wrist region of the patient;

FIG. 7 shows an exploded view of the catheter/IV cover of FIG. 6;

FIG. 8a provides a perspective view of a catheter/IV cover, modified, for application to a region of the hand; and

FIG. 8b provides a perspective view of the same catheter/IV cover in its flatten form.

### DESCRIPTION OF THE PREFERRED EMBODIMENT

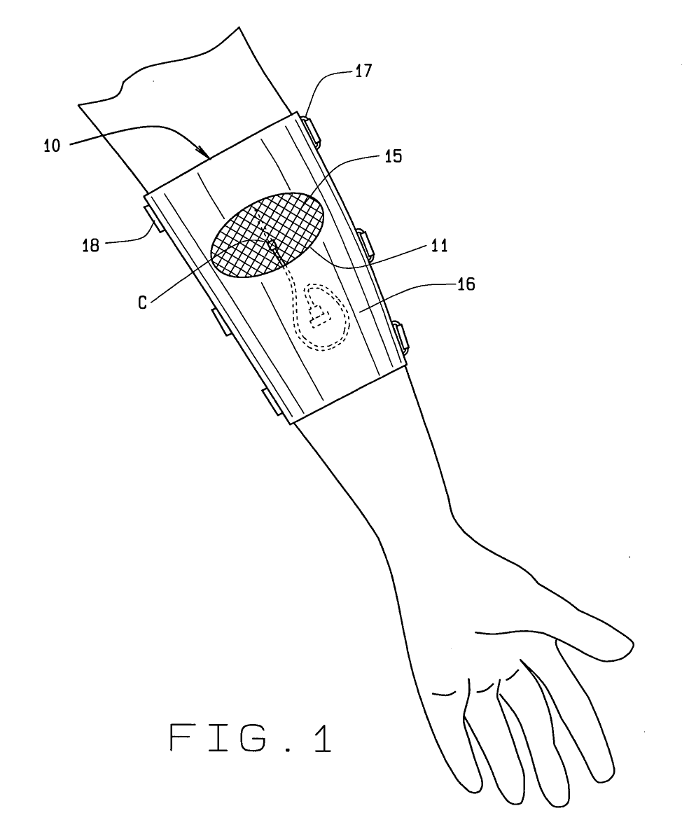

In referring to the drawings, and in particular FIG. 1, the catheter/IV cover 10 of this invention is readily disclosed. It is generally of a rectangular configuration, which can be applied to the upper surface of the arm in the region of the elbow, a portion of the arm therebelow, and to extend slightly above the elbow, into the bicep area of said arm. As noted, its rectangular configuration is such that it furnishes full coverage for this region of the arm, and provide the necessary grip of the Velcro®, where located. This can be used for a PICC line, which is a peripheral by inserted catheter/IV.

There is provided a cut out segment 11, which may be left open, for ease of viewing, or can have a mesh-like material applied therein, to add some transparency to the cover, and to apply pressure thereon, so that the catheter/IV C can be readily observed and that the catheter C can be readily maintained in place. This can also apply to a needle applicator. Preferably, though, the cut out segment will be of an oval configuration, as noted, and will be covered with a soft polyester tricot material, which is attached in place along the margins of the oval cut, so as to facilitate the bending of the arm when the catheter is in place, and the cover is overlying the same to secure the catheter components in place, at the same time, add to the comfort of the patient, but allow for the arm to be bent, at the elbow.

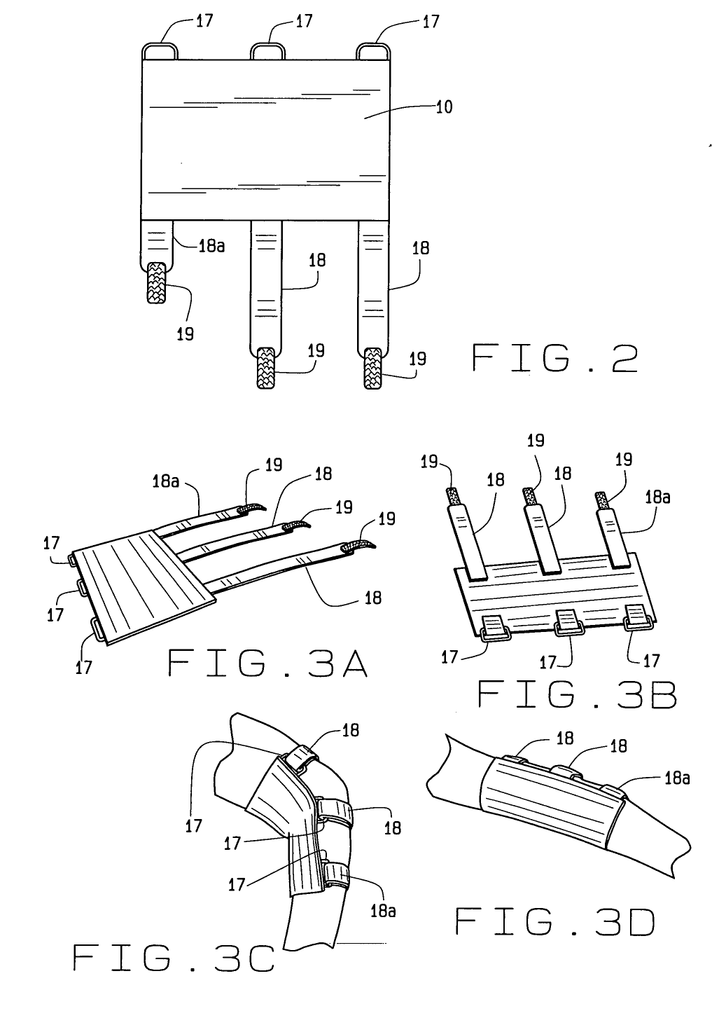

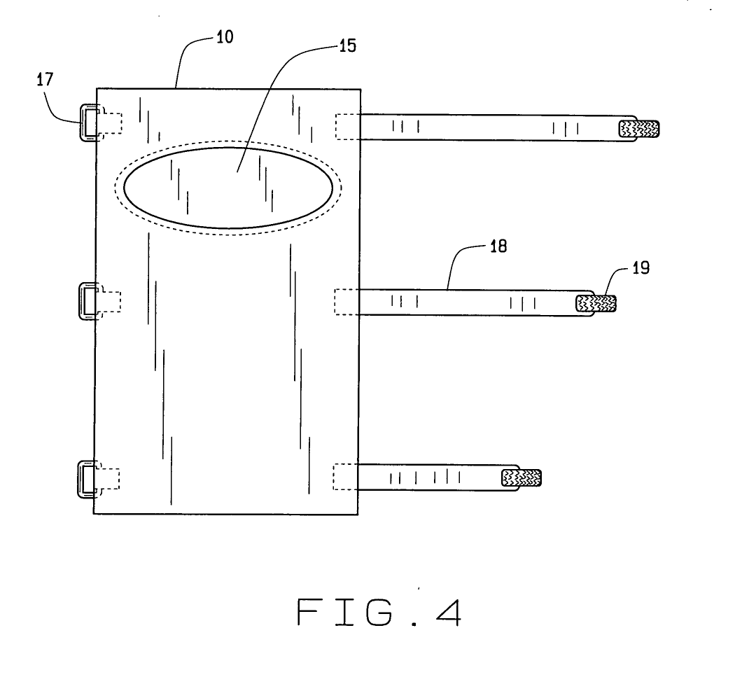

As can also be seen in FIGS. 2 through 5, there are tabs 12 that extend from the one side of the device, and include slits, as at 13, at the region where the tabs integrate with the cover 10, and these tabs and slits cooperate with the straps 18 that extend correspondingly through the slits, and the tabs 12 are bent over, and all of these components are stitched or glued in place, so as to hold the straps reinforced to the cover, when assembled. In addition, the tabs 14 extending from the opposite edge of the cover, are designed for looping around the ring loops 17, and it is these ring loops through which the straps 18 extend through and secure, by means of their Velcro® attachment, when the catheter/IV cover is held in place about the arm. The outer ends of the straps 18 may have guides, as at 19, fasten therewith, as also by stitching or gluing, or the like, and which are somewhat narrowed in configuration so as to facilitate their application through the loops or rings 17, when providing for securement of the cover about the arm.

It should be stated that the tabs 14 also extend through their associated loops 17, and then are folded over for stitching or gluing or otherwise attachment in place to the underside of the formed cover. It also needs to be stated that the straps may be formed of an elastic material, such as Velstretch®, available from Velcro Industries, of the Netherlands. This ads further elasticity to the cover, and its straps, when it is applied about the patient's arm, during usage.

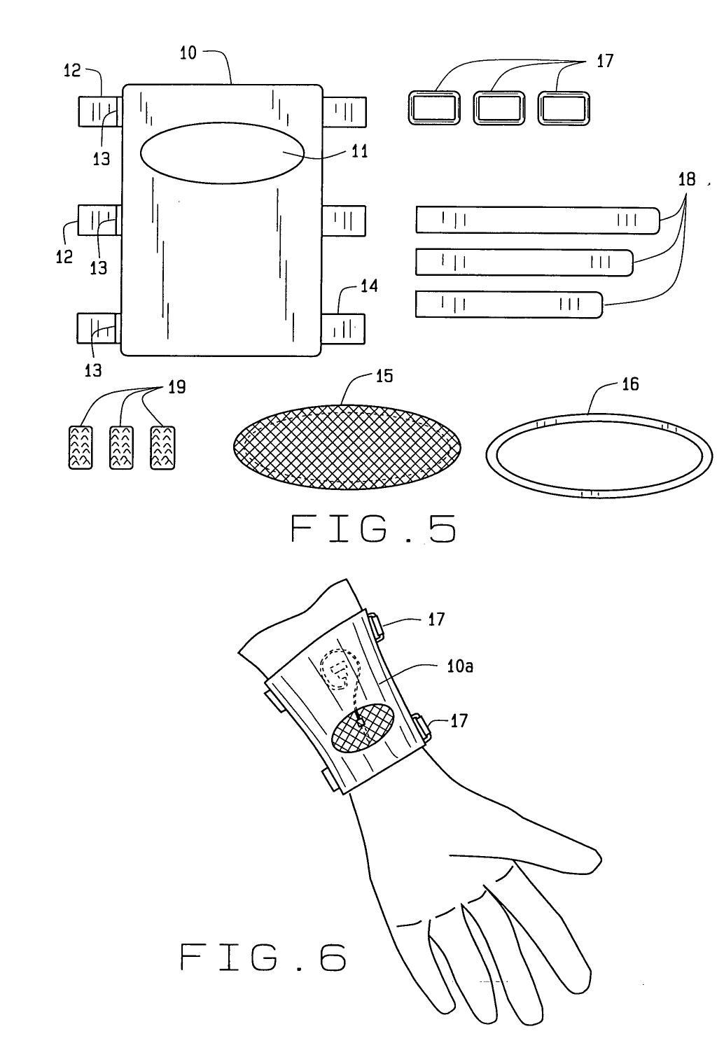

The oval opening 11, and which may be to any other configuration, includes a covering material, in the manner as previously described, and this can be seen in FIG. 5 at 15. It will be attached around its opening within the oval cut out 11, when applied within the structured cover. In addition, when a cover is applied in the oval cutout 11, it will be attached, stitched, or otherwise held in position about the edge of the opening, during its fabrication. There may even be a hemming material, as at 16 that may be applied around the periphery of the oval opening, in order to secure such cloth in place. This type of cloth material applied to the oval cut opening may comprise a window cover of flexible material, such as a polyester or netting, and which primarily adds flexibility to the cover, at the region of the elbow, so that the elbow can be easily bent, and not be obstructed by the catheter/IV cover at that location. The hemming material 16 may be fused, glued, or stitched in place, and may remain opened, to allow easy viewing of the catheter, when in place, or the polyester material 15 may be stitched, glued or fused in place, and it may provide softness and ease of bending of the cover, at that location, in the region of the elbow. And, as previously reviewed, it may be that the polyester material may be formed of a mesh, or other transparent like cloth material, in order to add to the viewing of the catheter in place, but at the same time, assure that the catheter is covered, and secure it against inadvertent access, when the cover is applied in place over an installed catheter.

FIGS. 2 and 3 also show the catheter/IV cover 10, with their square ring loops 17 applied to one side, and the straps 18 extending from the opposite side. In addition, the extension tabs 19 provide a narrowing section that may easily be guided through the loops 17, and these tabs may also be formed of Velcro®, to provide for rapid securement about the loops when fastened in place. The straps 18 themselves may be formed of a resilient or stretchable material, made of an elastic, such as Velstretch®, so that they will slightly give to provide for their snug fitting about the arm, and provide the necessary grip of the Velcro® when located. In addition, the lower strap, as noted at 18a, may be slightly shortened, requiring additional stretch, so as to furnish a very secure and tight fit about the arm, to provide for the snug positioning of the entire cover in place, once installed.

As previously reviewed, the cloth material forming the cover 10 may be made of a slightly resilient material, and be of a softer grade, such as an Ultrasuede®, which can be purchased at most fabric stores in the United States. The purpose for this is to provide a little stretch in the resiliency of the cover, so that it can bias lightly against the catheter, and its components, to secure them in place, and to retard and resist against the unauthorized pulling of the tube, from its catheter, which may occur when the patient is a little more active, or if somebody nearby is inadvertent and pulls on the tube without knowing it. Hence, the cover will help keep the catheter and its tube in place, and prevent its untimely removal.

It needs to be commented at this time that the various components that make up the catheter/IV cover of this invention may be provided in various coloration, whether it be bright colors to add to the spirits of the patient, or any other color that may be appropriate under the circumstances. Or, various of the components may be of differing colors, coordinated, to add to the attractiveness of the catheter/IV cover, when used, or can be used to identify the type of treatment being given.

The catheter cover shown in FIG. 6 is of a slightly modified design, and is designed for being applied at the area of the wrist, as can be seen. The cover 10a again is formed of the related cloth material, and may be either square or rectangular in configuration, as can be seen in FIG. 7. Extending from the sides are at least one loop, such as the loops 17 as shown, and these may be either tabs, with a slit, as shown at 13, and through which the straps 18 may apply, or these may be simply plastic or other material ring loops, as shown at 17, and through which the straps 18 may locate, and be fastened upon themselves, as by means of Velcro®, buckles, or any other means of fastening. And, the straps include the extending tabs 19, for reasons as previously explained, which may facilitate guidance of the strap through the loop 17, when installed, and such tabs may be formed of Velcro®, to assure fastening of the stretchable like Velstretch® straps in place, once the device is assembled.

As can also be seen in FIG. 7, the cover 10a may include a pair of apertures, as at 10b, and through which fingers may be inserted, when the cover is to be used for application to the hand, as will be subsequently described. This includes the use of a heplock line for treatment.

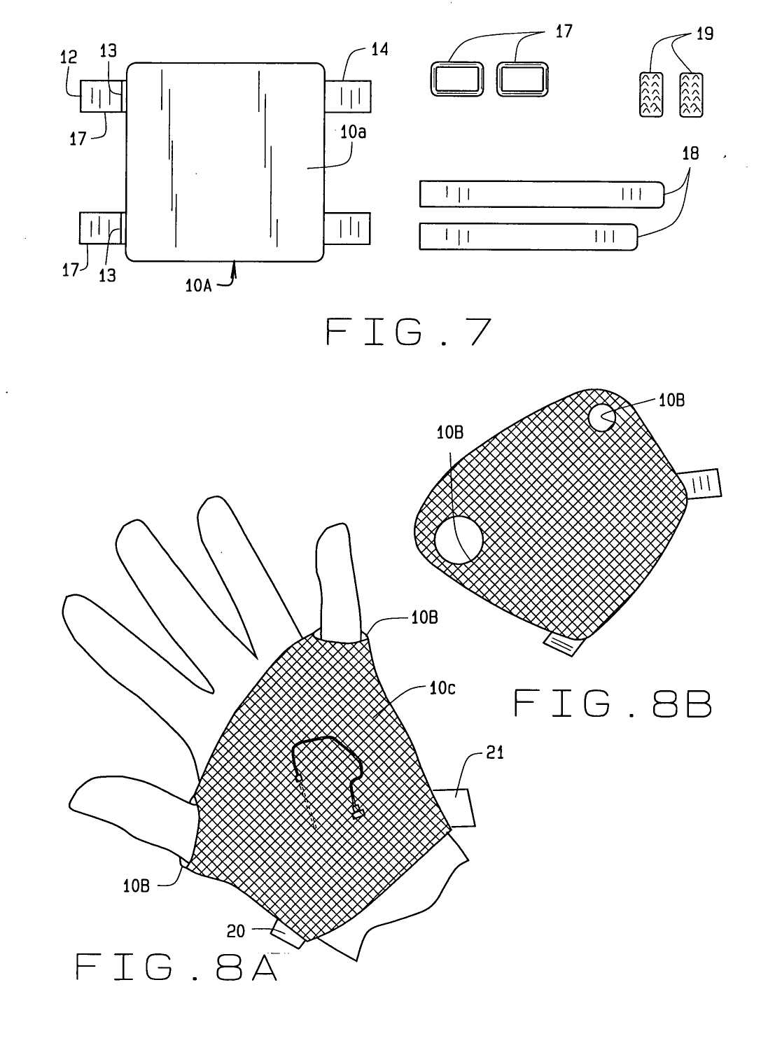

More specifically, the hand model of the catheter/IV cover is shown in FIG. 8. It likewise includes a covering material 10c, as noted. When in place, it can seen that it thoroughly covers the catheter C, and its tubing, when in place.

The cover is designed to provide for coverage over various parts of the hand, particularly the back of the hand, when the catheter is applied in this vicinity. The upper corners of the cover include a pair of apertures, or loops, as at 10b, and through which the thumb and small or little finger locate, for positioning of the catheter/IV cover onto the hand. Then, the bottom edges of the cover include the shown loop 20, and corresponding strap 21, which may be fastened together in the manner as previously explained and reviewed with respect to the other forms of catheter/IV covers as embodied in this invention.

It is just as likely that the cover this invention may be applied to various other parts of the body, and act as a catheter/IV cover, as such a cover may also be applied not only to adults, but also to children. There is also the remote possibility that these types of covers could be used for holding a form of catheter in place, even in the animal veterinary field, where such a cover maybe essential for holding a catheter in place.

In summary, the catheter/IV covers of this invention are provided and constructed of the soft type material like Ultrasuede®, as described. The purposes for this have already been reviewed. They may be of bright or multiple colors, to add to their attractiveness. Preferably, the materials from which the cover is made will be washable, so they are capable of being reused by the patient, during treatment. Furthermore, the design of these covers indicate that they are of reasonably small size, can be folded into smaller sizes, for storage, or may even be applied into one's pocket, as when not in use. These are all advantages of this type of a cover as assembled. This particular catheter/IV cover is primarily for use with the ambulatory patient, when being moved from one location to another, and the purpose for the catheter may be temporarily disconnected. Hence, this cover provides a full cover for the catheter, and prevents a disruption to use of the catheter, or any inadvertent removal.

Variations or modifications to the subject matter of this invention may occur to those skilled in the art upon review of the invention as described herein. Such variations, if within the spirit of this development, are intended to be encompassed within the scope of invention provided herein. The depiction of the invention in the summary, and the detailed disclosure in the description of the preferred embodiment, and as shown in the drawings, are set forth for illustrative purposes only.

1. A catheter/IV cover for use for covering one of the arm in the region of the elbow, the wrist, or the hand, said catheter/IV cover including a covering material, of a shape to provide for overlying coverage for at least one half of the arm, wrist, or hand, a fastening device securing with each lateral edge of the covering device, at least one strap extending from one lateral side edge, and at least one loop extending from the opposite lateral edge, the strap provided for engagement through the loop to secure the catheter/IV cover in place, the catheter/IV cover being fabricated of a soft and pliable material so as to provide minor stretch when biasing against the catheter and its tube when in place, and the strap having sufficient elasticity and resiliency to provide for the force necessary to hold the catheter, tube, and the overlying cover when the cover is secured during medical treatment and ambulation of the patient.

2. The catheter/IV cover of claim 1 wherein said cover is provided for application to the hand, and the upper corner of said cover having apertures therethrough for engagement with select fingers of the hand, while the strap and loop secure with the bottom lateral edges of the cover for fastening when the cover is used for overlying a catheter applied to the hand.

3. The catheter/IV cover of claim 1 and including an aperture provided through the catheter/IV cover, said aperture furnishing a weakened area to allow for bending of the cover when it is applied approximate the elbow of the patient, and to allow for viewing of the emplaced catheter during its application.

4. The catheter/IV cover of claim 3 and including a light flexible material engaged within the aperture opening of the cover to provide a mild biasing force against the catheter when in place, and to allow the elbow of the arm to be pivoted, during usage.

5. The catheter/IV cover of claim 4 wherein the cloth material applied to the cover aperture is a flexible mesh.

6. The catheter/IV cover of claim 5 wherein the mesh is at least partially transparent.

7. The catheter/IV cover of claim 1 wherein the cover with its straps provides a slight biasing force upon both the catheter and its associated tubing during their application.

8. A catheter/IV cover for use for covering a limb or other component of a patient during treatment, the catheter/IV cover including a covering material, of a shape to provide for overlying coverage of at least a contiguous portion of the body, a fastening device securing with the edge of the covering device, said fastening device provided for securement of the catheter/IV cover to the proximate body part, said catheter/IV cover being fabricated of a soft and pliable material so as to provide minor stretch when biasing against the catheter and its tube when in place, and the fastening device having sufficient elasticity in resiliency to provide for the force necessary to hold the catheter, tube, and overlying covering when the cover is secured during medical treatment.

9. The catheter/IV cover of claim 8 and including an aperture providing through the catheter/IV cover, said aperture furnishing a weakened are to allow for bending of the cover when it is applied approximate the patient body part, and to allow for viewing of the emplaced catheter during its application.

10. The catheter/IV cover of claim 9 and including a cloth material applied to the cover aperture to provide it with closure.

11. The catheter/IV cover of claim 10 were in the cloth material applied to the cover aperture is a flexible mesh.
